(19) United States
(12) Patent Application Publication
(10) Pub. No.: US 2009/0258947 A1
(43) Pub. Date: Oct. 15, 2009

(54) NOVEL LOW DOSE PHARMACEUTICAL COMPOSITIONS COMPRISING NIMESULIDE, PREPARATION AND USE THEREOF

(76) Inventors: Rajesh Jain, New Delhi (IN); Kour Chand Jindal, New Delhi (IN)

Correspondence Address: LADAS & PARRY LLP, 26 WEST 61ST STREET, NEW YORK, NY, 10023, US

(21) Appl. No.: 12/298,054

(22) PCT Filed: Apr. 23, 2007
(86) PCT No.: PCT/IN07/00162

§ 371 Date: Oct. 22, 2008

(30) Foreign Application Priority Data
Apr. 24, 2006 (IN) 1033/DEL/2006

(51) Int. Cl. A61K 31/16; A61P 25/00

(52) U.S. Cl. 514/608

(57) ABSTRACT

Low dose pharmaceutical dosage form comprising nimesulide or its pharmaceutically acceptable salts, esters, solvates or hydrates thereof, along with one or more pharmaceutically acceptable excipient(s) are provided. The present invention also provides process of preparing such dosage forms and therapeutic methods of using such dosage forms. The low dose compositions 10 are designed to exhibit such bioavailability, which is effective in the treatment of NSAID indicated disorders particularly, which require long-term treatment regimens such as arthritis. Such compositions reduce the cost of therapy in diseases, which require long-term therapies, are easy to manufacture, and also result in the reduction of dose related side effects associated with nimesulide therapy.

### FIELD OF THE INVENTION

The present invention relates to novel low dose pharmaceutical dosage form comprising nimesulide or its pharmaceutically acceptable salts, esters, prodrugs, solvates, hydrates, or derivatives thereof, along with one or more pharmaceutically acceptable excipients(s). The present invention also provides process of preparing such dosage form and therapeutic methods of using such dosage form. The low dose compositions are designed to exhibit such bioavailability, which is effective in the treatment of NSAID indicated disorders particularly those that require long-term treatment regimens such as arthritis. Such compositions reduce the cost of therapy in diseases, which require long-term therapies, are easy to manufacture, and also results in the reduction of dose-related side effects associated with nimesulide therapy.

### BACKGROUND OF THE INVENTION

Cyclooxygenase-1 (COX-1) is an enzyme, which is normally present in a variety of areas of the body including sites of inflammation and the stomach. The COX-1 enzyme of the stomach produces certain chemical messengers (called prostaglandins) that ensure the natural mucus lining which protects the inner stomach. Common anti-inflammatory drugs like aspirin block the function of the COX-1 enzyme along with another enzyme, COX-2. When the COX-1 enzyme is blocked, inflammation is reduced, but the protective mucus lining of the stomach is also reduced, which can cause stomach upset, ulceration and bleeding from the stomach and intestines.

Cyclooxygenase-2 (COX-2) inhibitors are newly developed drugs for inflammation that selectively block the COX-2 enzyme. Blocking this enzyme impedes the production of the chemical messengers (prostaglandins) that cause the pain and swelling of arthritis inflammation. COX-2 inhibitors are a new class of nonsteroidal anti-inflammatory drugs (NSAIDs). Because they selectively block the COX-2 enzyme and not the COX-1 enzyme, these drugs are uniquely different from traditional NSAIDs; This selective action provides the benefits of reducing inflammation without irritating the stomach.

These drugs pose an advantage in comparison to previous anti-inflammatory drugs because their mechanism of action carries nowhere near the risk of stomach ulceration and bleeding. The COX-2 inhibitors include celecoxib, rofecoxib, etoricoxib, valdecoxib, itacoxib, deracoxib and the like.

Nonsteroidal anti-inflammatory drugs (NSAIDs) are commonly prescribed medications for the inflammation of arthritis and other body tissues, such as in tendinitis and bursitis. Examples of NSAIDs include aspirin, indomethacin, nimesulide, ketorolac, diclofenac, ibuprofen, naproxen, piroxicam, nabumetone, and the like. Nimesulide is a potent NSAID, presently used in the treatment of painful inflammatory conditions, due to rheumatoid arthritis, which also possesses antipyretic activity. Compared to other NSAIDs, nimesulide has a better therapeutic ratio, low gastrotoxicity and generally good tolerability.

Nimesulide (4-nitro-2-phenoxymethane-sulfonanilide) is a NSAID that is weakly acidic (pKa 6.5) and differs from other NSAIDs in that its chemical structure contains a sulfonanilide moiety as the acidic group. It has good anti-inflammatory, analgesic and antipyretic activity, and is well tolerated by patients. It is conventionally orally administered as 100 mg or 200 mg b.i.d. tablets. Nimesulide is the first marketed drug with a selective inhibition of prostaglandin synthesis via cyclooxygenase-2 (COX-2), which results in lower toxicity in the gastrointestinal mucosa and the kidney. The safety aspects relating to the stomach and kidney are particularly important in comparison with other NSAIDs. Nimesulide administered orally to healthy volunteers is rapidly and extensively absorbed, regardless of the presence of food. After oral administration of a 50 mg dose, the mean peak plasma concentrations (Cₘₐₓ) ranged from 1.98 to 2.30 with a time to reach Cₘₐₓ(tₘₐₓ) from 2.51 to 3.00 hours. Following oral administration of a 100 mg dose to healthy fasting volunteers, Cₘₐₓ of 2.86 to 6.50 mg/L occurred within 1.22 to 2.75 hours after administration. Nimesulide concentrations of approximately 25 to 80% of Cₘₐₓ appeared at the first sampling time (30 minutes) after administration. With regard to the area under the plasma concentration-time curve (AUC), after a single oral dose of nimesulide 100 mg in fasting individuals, AUC ranged from 14.65 to 54.09 mg·h/L. Time to reach peak concentration (tₘₐₓ) ranged from 1 to 4 hours after 100 mg and from 1 to 6 hours after 200 mg dose, the respective means being 2.50 and 3.17 hours. The estimated mean terminal elimination half-life (t_{1/2z}) varies from about 1.80 to 4.73 hours. Nimesulide is chiefly eliminated by metabolic transformation and the main metabolite is 4′-hydroxy-nimesulide. After single-dose administration of nimesulide 100 mg, the Cₘₐₓ of the 4-hydroxy-derivative ranged from 0.96 to 1.57 mg/L and was attained within 2.61 to 6.33 hours (tₘₐₓ), i.e. 1 to 3 hours later than that of the parent compound. Compared to other non-steroidal anti-inflammatory drugs, nimesulide has a favorable therapeutic index, minimal acute gastrointestinal toxicity and generally exhibits a good tolerability.

Various formulations are available in the market comprising 50 mg of nimesulide for twice-a-day administration particularly as a pediatric dose primarily in the form of 50 mg/5 ml suspension or 50 mg kid tablets such as those available under the brand name Nimulid®. Also fixed dose combinations such as combination of 50 mg nimesulide with 125-500 mg paracetamol are available. However, no composition is yet available which comprises low dose nimesulide preferably for adult use, wherein the total daily dose of nimesulide is less than the conventionally administered daily dose of at least about 200 mg of nimesulide, and which is still effective for the treatment of several cyclooxygenase enzyme inhibition mediated or NSAID indicated disorders.

Nimesulide is a very hydrophobic drug substance and is practically insoluble in water. Its aqueous solubility is about 0.01 mg/ml at room temperature. The very poor aqueous solubility and wettability of the drug present problems for the preparation of pharmaceutical formulations with good release and non-variable bioavailability. It is desirable to overcome the disadvantages connected with the poor aqueous solubility and wettability of nimesulide. One possible method is the reduction in the dose of nimesulide, thus leading to a reduction in the hydrophobic content of the composition, which is an objective of the present invention.

U.S. Pat. No. 6,017,932 describes pharmaceutical composition having increased therapeutic efficacy comprising at least one NSAID selected from the group consisting of nimesulide, nabumetone, tepoxalin, and flosulide as active ingredient and a bioavailability enhancer such as piperine.

However, still there exists a need to develop pharmaceutical compositions comprising low dose of nimesulide, wherein the total daily dose of nimesulide is less than the conventionally administered daily dose of at least about 200 mg of nimesulide, which does not require the use of any specific bioenhancer and which can still release the drug in a desired manner and in a quantity sufficient to alleviate desired pathological conditions without causing or at least minimizing dose related toxicity, and can be prepared in an easy and cost-effective manner.

The inventors of the present invention have done extensive research and conducted several experiments to alleviate the drawbacks existing in the prior art to develop dosage form compositions comprising a low dose of nimesulide that has reduced side effects and are easy to formulate by using different conventional excipients, thus demonstrating a significant advancement over the prior art.

### SUMMARY OF INVENTION

It is the objective of the present invention to provide novel low dose pharmaceutical dosage form comprising nimesulide or its pharmaceutically acceptable salts, esters, prodrugs, solvates, hydrates, or derivatives thereof, along with one or more pharmaceutically acceptable excipient(s).

It is also an objective of the present invention to provide novel low dose pharmaceutical dosage form comprising nimesulide wherein the total daily dose of nimesulide is less than the conventionally administered daily dose of at least about 200 mg of nimesulide. It is also an objective of the present invention to provide novel low dose pharmaceutical dosage form comprising nimesulide, wherein the dose of the nimesulide is in an amount below 200 mg, for a single administration intended for once-a-day administration, and wherein the total daily dose of nimesulide is less than the conventionally administered daily dose of at least about 200 mg of nimesulide.

It is also an objective of the present invention to provide novel low dose pharmaceutical dosage form comprising nimesulide, wherein the individual dose of the nimesulide is in an amount below 100 mg, for a single administration intended for twice-a-day administration, and wherein the total daily dose of nimesulide is less than the conventionally administered daily dose of at least about 200 mg of nimesulide.

It is also an objective of the present invention to provide novel low dose pharmaceutical dosage form comprising nimesulide additionally with one or more other active agent(s) whose concurrent administration may be useful for the treatment of one or more pathological condition(s).

It is yet another objective of the present invention to provide process of preparation of such dosage form comprising low dose of nimesulide or its pharmaceutically acceptable salts, esters, prodrugs, solvates, hydrates, or derivatives thereof which comprises treating nimesulide with one or more pharmaceutically acceptable excipient(s), optionally adding one or more other active agent(s), and formulating into the suitable dosage form.

It is also an objective of the present invention to provide use of the low dose pharmaceutical dosage form comprising nimesulide for the management of cyclooxygenase enzyme mediated disorders and/or cyclooxygenase inhibitor indicated disorders which comprises administrating to a subject in need thereof a pharmaceutically effective amount of the composition.

It is also an objective of the present invention to provide use of the low dose pharmaceutical dosage form comprising nimesulide for the prophylaxis, amelioration and/or treatment of cyclooxygenase enzyme mediated disorders and/or cyclooxygenase inhibitor indicated disorders which comprises administrating to a subject in need thereof a pharmaceutically effective amount of the composition. The low dose compositions are designed to exhibit such bioavailability, which is effective in the treatment of NSAID indicated disorders particularly, which require long term treatment regimens such as arthritis. Such compositions reduce the cost of therapy in diseases, which require long-term therapies, and also results in the reduction of dose-related side effects associated with nimesulide therapy.

Thus, it is yet another objective of the present invention to provide low dose pharmaceutical dosage form, wherein the dosage form is capable of providing therapeutically effective bioavailability of nimesulide with reduced side effects, after dosing in a human subject.

### DETAILED DESCRIPTION OF THE INVENTION

Nimesulide is conventionally given as 100-200 mg tablets or capsules b.i.d. (twice-a-day) or 50 mg/ml suspension for the treatment of inflammatory disorders, pain, arthritis and the like. High dosage compositions of nimesulide are associated with dose related side effects such as gastrotoxicity or liver toxicity or even some cardiac disorders or any other disorders arising due to cyclooxygenase enzyme inhibition. The inventors of the present invention have made an effort to alleviate or at least reduce the dose related side effects associated with nimesulide by reducing the dose of nimesulide conventionally administrated. Furthermore, the low dose compositions have improved solubility and in turn improved bioavailability, and are easy to formulate. Further, such novel compositions require lesser quantity of excipients and thus are preferably smaller in size compared to the conventionally available dosage forms, which in turn leads to better patient acceptability. Preferably, the compositions of the present invention do not require the use of any specific bioenhancer or the like.

The term ‘low dose’ as used herein refers to the therapeutically effective dose of nimesulide which is less than the usual or the conventional dose required to produce the therapeutic effect.

The present invention provides novel low dose pharmaceutical dosage form compositions comprising nimesulide or its pharmaceutically acceptable salts, esters, prodrugs, solvates, hydrates, or derivatives thereof, along with one or more pharmaceutically acceptable excipient(s). Preferably the compositions comprise nimesulide either intended to be administered once-a-day or twice-a-day in a dose less than the conventionally administered adult oral dose, which is about 100 mg of nimesulide twice-a-day in the tablet form or in a dose less than the conventionally administered pediatric oral dose, which is about 50 mg of nimesulide twice-a-day in the tablet form.

In an embodiment of the present invention, the daily dose of nimesulide is lower than the conventional recommended dose for the treatment of long term NSAID indicated disorders. In another embodiment is provided a low dose pharmaceutical dosage form composition comprising nimesulide, wherein the total daily dose of nimesulide is less than the conventionally administered daily dose of at least about 200 mg of nimesulide.

In another embodiment of the present invention is provided novel low dose pharmaceutical dosage form comprising nimesulide wherein the individual dose of the nimesulide is in an amount below 100 mg, for a single administration, intended for twice-a-day administration, and wherein the total daily dose of nimesulide is less than the conventionally administered daily dose of at least about 200 mg of nimesulide. The individual dose of nimesulide is preferably ranges from about 10 to about 95 mg, and more preferably the individual dose of nimesulide ranges from about 25 to about 85 mg, for a single administration, intended for twice-a-day administration.

In an embodiment of the present invention the low dose pharmaceutical dosage form of nimesulide comprises the dose of the nimesulide in an amount below 200 mg, intended for once-a-day administration, preferably the dose may ranges from about 125 mg to about 180 mg.

In another embodiment of the present invention the low dose pharmaceutical dosage form of nimesulide comprises the dose of the nimesulide in an amount below 200 mg, intended for once-a-day administration, wherein the low dose of the nimesulide intended for once-a-day administration is administered either in single unit or in a multiple unit. Particularly, the low dose of the nimesulide intended for once-a-day administration is administered in a single unit, preferably in the form of a tablet.

In an embodiment, the bioavailability and in turn the plasma concentration of nimesulide present in the novel composition of the present invention is sufficient to produce desired pharmacological effects such as analgesic and/or anti-inflammatory and/or antipyretic effects and the like. Particularly, the low dose pharmaceutical dosage form of the present invention is capable of providing therapeutically effective bioavailability of nimesulide with reduced side effects, after dosing in a human subject.

Particularly the composition of the present invention is very useful in mammals, more particularly in humans, for the treatment of preferably the NSAID indicated disorders such as acute painful conditions like lower back pain, early morning pathologies, post-operative trauma, pain associated with cancer, postoperative pain, sports injuries, dysmenorrhoea, migraine headache, neurological pain and pain associated with sciatica and spondylitis, arthritis, idiopathic pain, myofascial pain, osteoarthritis, neuropathic pain, fibromyalgia and inflammatory pain states such as rheumatoid arthritis and osteoarthritis. Neuropathic pain includes pain such as pain secondary to injury to nerves and includes postherpetic neuralgia, diabetic neuropathy, postamputation pain, mono- and polyneuropathies, radiculopathy, central pain, shingles, trigeminal neuralgia, temporomandibular joint disorder; cancer pain; chronic pain; sympathetically mediated pain, Raynaud's disease, CPS (Chronic Pain Syndrome); tension and migraine headache, stump pain, polyarteritis nodosa, osteomyelitis, burns involving nerve damage, AIDS related pain syndromes, and connective tissue disorders, such as systemic lupus erythematosis, systemic sclerosis, polymyositis, and dermatomyositis, other degenerative joint disorders, or any other disorders mediated by particularly the cyclooxygenase enzyme, and the like, or a combination of several disorders or any other associated disorder(s). Also, the low dose compositions comprising nimesulide are useful as an antioxidant or a platelet aggregation inhibitor or as an anticancer agent.

In an embodiment, the compositions of the present invention comprises reduced dose of nimesulide but are still prophylactically or therapeutically effective, and hence provides a reduction in the cost of therapy in diseases like arthritis which require long term therapies. The low dose compositions of nimesulide also result in the reduction of dose related side effects associated with NSAID therapy.

In yet another embodiment of the present invention, the low dose nimesulide is present alongwith at least one another active agent(s) selected from but not limited to a group comprising antipyretics such as acetaminophen, antiallergics such as cetirizine or loratadine or fexofenadine, aldosterone receptor antagonists, antibiotics, various enzymes, antimuscarinic agents, anti-viral agents, protein kinase inhibitors, α2-adrenergic agonist, ACE inhibitors, opioid analgesics, steroids, leukotriene B₄(LTB₄) receptor antagonists, leukotriene A₄ (LTA₄) hydrolase inhibitors, 5-HT agonists, HMG CoA inhibitors, H₂ antagonists, antineoplastic agents, antiplatelet agents, thrombin inhibitors, decongestants, diuretics, sedating or non-sedating anti-histamines, inducible nitric oxide synthase inhibitors, opioids, analgesics, Helicobacter pylori inhibitors, bronchodilators, spasmolytics such as scopolamine or glucagon, muscle relaxants, proton pump inhibitors, isoprostane inhibitors, PDE4-inhibitors, other NSAIDs, selective or preferential COX-2 inhibitors, COX-1 inhibitors, expectorants such as bromohexine and pseudoephedrine, analgesics such as codeine and chlorzoxazone and mefenamic acid and tramadol, antiemetics, urinary acidifiers such as racemethionine, chondroitin, glucosamine, methyl sulfonyl methane (MSM), aspirin, antidepressants, antipsychotics, antimigraine agents, and the like or mixtures thereof.

The novel derivatives of the present invention can easily be formulated into desired pharmaceutical compositions, which can be administered orally, parenterally, topically, transdermally, rectally or by any other route of administration. In a further embodiment, the composition of the present invention is preferably in the form of oral dosage forms such as powder, granules, tablets, capsules, pellets, suspensions, solutions, emulsions, or the like, more preferably as a solid oral dosage form such as tablets or capsules. The tablets can be prepared by either wet granulation, direct compression, or by dry compression (slugging). In a preferred embodiment of the present invention, the oral composition is prepared by wet granulation. The granulation technique is either aqueous or non-aqueous. The non-aqueous solvent used is selected from a group comprising acetone, ethanol, isopropyl alcohol or methylene chloride. In an embodiment, the compositions of the present invention are in the form of compressed tablets, moulded tablets, mini-tablets, capsules, pellets, granules and products prepared by extrusion or film cast technique, and the like. The tablets/minitablets may be optionally coated with a nonfunctional coating to form a nonfunctional layer. The tablets/minitablets may be optionally filed into capsules. In another embodiment, the pharmaceutical composition may contain other pharmacologically active ingredient(s) whose concurrent administration may be useful.

In an embodiment, the pharmaceutically acceptable excipient of the present invention preferably comprises a polymeric material selected from but not limited to the group comprising pH dependent polymers; pH independent polymers; swellable polymers; hydrophilic polymers; hydrophobic polymers and/or one or more other hydrophobic materials; ionic polymers such as sodium alginate, carbomer, calcium carboxymethylcellulose or sodium carboxymethylcellulose; non-ionic polymers such as hydroxypropyl methylcellulose; synthetic or natural polysaccharide selected from the group comprising alkyl celluloses, hydroxyalkyl celluloses, cellulose ethers, cellulose esters, nitrocelluloses, dextrin, agar, carrageenan, pectin, furcellaran, starch and starch derivative, and mixtures thereof. The polymeric material used in the present invention is selected from but not limited to a group comprising cellulosic polymer, methacrylate polymer, PVP, alginate, PVP-PVA copolymer, ethylcellulose, cellulose acetate, cellulose propionate (lower, medium or higher molecular weight), cellulose acetate propionate, cellulose acetate butyrate, cellulose acetate phthalate, cellulose triacetate, poly(alkyl methacrylate), poly(isodecyl methacrylate), poly(lauryl methacrylate), poly(phenyl methacrylate), poly(alkyl acrylate), poly(octadecyl acrylate), poly(ethylene), poly(alkylene), poly(alkylene oxide), poly(alkylene terephthalate), poly(vinyl isobutyl ether), poly(vinyl acetate), poly(vinyl chloride) and polyurethane or a mixture thereof used either alone or in combination. In an embodiment, the composition additionally comprises one or more pharmaceutically acceptable excipient(s) selected from gum, surfactant and complexing agent.

In a further embodiment, the gum useful in the present invention is selected from but not limited to a group comprising xanthan gum, guar gum, gum arabic, carrageenan gum, karaya gum, locust bean gum, acacia gum, tragacanth gum, agar and the like or mixtures thereof. In another embodiment, the surfactant useful in the present invention is selected from a group comprising anionic surfactants, cationic surfactants, non-ionic surfactants, zwitterionic surfactants or mixtures thereof. In yet another embodiment, the complexing agent useful in the present invention is a cyclodextrin selected from a group comprising but not limited to alpha-cyclodextrin, beta-cyclodextrin, betahydroxy-cyclodextrin, gamma-cyclodextrin, and hydroxypropyl cyclodextrin, or the like, or any other complexing agent known to the art.

In another embodiment is provided a process for the preparation of novel low dose composition of nimesulide, which comprises of the following steps:
i) treating nimesulide with one or more pharmaceutically acceptable excipient(s),
ii) optionally adding one or more other active agent(s), and
iii) formulating into a suitable dosage form.

In a further embodiment of the present invention, the pharmaceutical composition can be prepared by well known methods in the art, e.g. by mixing nimesulide with one or more pharmaceutical excipient(s) optionally with another active agent(s). The solid dosage forms can be produced by known methods such as direct compression, granulation, compaction, extrusion, molding, or the like using conventional excipients. For semi-solid or liquid preparations, in additional to a solid excipients, liquid and/or semi-solid excipients known to the art are used. For the preparation of an injectable composition such as an intra-venous or intra-muscular injection, the novel derivatives are treated with pharmaceutical excipients such as solvents, buffers, and the like, known to a person skilled in art.

The pharmaceutically acceptable excipients useful in the composition of the present invention are selected from but not limited to a group of excipients generally known to persons skilled in the art such as fillers, binders, disintegrants, glidants, lubricants, colorants; stabilizers; preservatives; chelating agents; vehicles; bulking agents; stabilizers; preservatives; hydrophilic polymers; solubility enhancing agents such as glycerine, various grades of polyethylene oxides, transcutol and glycofurol; tonicity adjusting agents; local anesthetics; pH adjusting agents; antioxidants; osmotic agents; chelating agents; viscosifying agents; wetting agents; emulsifying agents; acids; sugar alcohol; reducing sugars; non-reducing sugars and the like used either alone or in combination thereof e.g. diluents such as lactose, mannitol, sorbitol, starch, microcrystalline cellulose, xylitol, fructose, sucrose, dextrose, dicalcium phosphate, calcium sulphate; bulking agent and organic acid(s). The disintegrants used in the present invention include but not limited to starch or its derivatives, partially pregelatinized maize starch (Starch 1500®), croscarmellose sodium, sodium starch glycollate, and the like used either alone or in combination thereof. The lubricants used in the present invention include but not limited to talc, magnesium stearate, calcium stearate, stearic acid, hydrogenated vegetable oil and the like used either alone or in combination thereof. The vehicles suitable for use in the present invention can be selected from but not limited to a group comprising dimethylacetamide, dimethylformamide and dimethylsulphoxide of N-methylpyrrolidone, benzyl benzoate, benzyl alcohol, ethyl oleate, polyoxyethylene glycolated castor oils (Cremophor® EL), polyethylene glycol MW 200 to 6000, propylene glycol, hexylene glycols, butylene glycols and glycol derivatives such as polyethylene glycol 660 hydroxy stearate (commercially available as Solutrol® HS15). In another embodiment of the present invention, the compositions may additionally comprise an antimicrobial preservative such as benzyl alcohol preferably at a concentration of 2.0% v/v of the composition. It will be appreciated that certain excipients used in the present composition can serve more than one purpose. In an embodiment of the present invention, the composition may additionally comprise a conventionally known antioxidant such as ascorbyl palmitate, butyl hydroxy anisole, butyl hydroxy toluene, propyl gallate and α-tocopherol.

In another embodiment, the composition of the present invention can be formulated into a dosage form selected from the group consisting of tablets, capsules, liquid dispersions, oral suspensions, gels, aerosols, ointments, creams, fast melt formulations, lyophilized formulations, injectables, controlled release formulations, delayed release formulations, extended release formulations, pulsatile release formulations, and mixed immediate release and controlled release formulations. Preferably the low dose dosage form is formulated in the form of tablet, which may be coated with one or more functional or non-functional coating layers.

In another embodiment of the present invention is provided an use of the dosage form composition comprising low dose of nimesulide for the treatment of NSAID indicated disorders which comprises administrating to a subject in need thereof a pharmaceutically effective amount of the composition. In a further embodiment of the present invention, is provided an use of the dosage form for the management which includes prophylaxis, amelioration and/or treatment of particularly pain and/or inflammation associated with osteoarthritis, ligamentous pain, bursitis, tendinitis, low back pain, postoperative pain, dental extraction or surgery; saphenectomy or inguinal hernioplasty; haemorrhoidectomy; acute musculoskeletal injury; ear, nose or throat disorders; gynecological disorders; cancer pain; Alzheimer's disease; thrombophlebitis; urogenital disorders; bursitis or tendonitis; morning stiffness associated with rheumatoid arthritis, idiopathic pain, myofascial pain, osteoarthritis, neuropathic pain, fibromyalgia and inflammatory pain states such as rheumatoid arthritis and osteoarthritis. Neuropathic pain includes pain such as pain secondary to injury to nerves and includes postherpetic neuralgia, diabetic neuropathy, postamputation pain, mono- and polyneuropathies, radiculopathy, central pain, shingles, trigeminal neuralgia, temporomandibular joint disorder; cancer pain; chronic pain; acute pain; breakthrough pain sympathetically mediated pain, Raynaud's disease, CPS (Chronic Pain Syndrome); tension and migraine headache, stump pain, polyarteritis nodosa, osteomyelitis, burns involving nerve damage, AIDS related pain syndromes, and connective tissue disorders, such as systemic lupus erythematosus, systemic sclerosis, polymyositis, and dermatomyositis, other degenerative joint disorders and the like.

In an embodiment, the low dose compositions comprising nimesulide is particularly useful for the treatment of such NSAID indicated disorders which are particularly chronic in nature and which require a long term but mild to moderate treatment, or even some acute conditions which favorably respond to or are alleviated by a low dose of nimesulide. The low dose compositions can be used prophylactically or therapeutically depending on the pathological condition intended to be prevented or treated respectively.

In another embodiment, the low dose compositions of the present invention are useful in the management of inflammation and pain and one or more other associated disorders such as gastric ulcer, intermittent or episodic pain, angiogenesis, viral infections, cardiovascular diseases, neoplasia, cancer, urinary incontinence, bacterial infections, arthritis, migraine, asthma, and like.

### Pharmacological Studies:

A pharmacological study has been carried out to study the antiinflammatory effect of low dose of nimesulide in carrageenan induced paw edema in Wistar rats. Male Wistar rats (180-250 g) were selected for the study comprising 6 animals in each group for the duration of study of 1 day. The vehicle used was (0.5% CMC in 0.1% Tween®80) and the route of administration was peroral (p.o.). The overnight fasted rats were orally administered with various doses of nimesulide with 5 ml/kg of normal saline. One hour later 0.1 ml of 1% carrageenan was administered s.c. to the right hind paw of the rat. The degree of inflammation was measured by using digital plethosmometer (Cat. No. 7140, Ugo Basile, Italy) at 0 (initial), 0.5, 1, 2, 3 and 5 hours (h) post carrageenan injection and the values were recorded. For each hour, at least 2-3 values of the dipped paw were obtained and later averaged. The increase in the paw volume (ml) was calculated by subtracting the values obtained at the n^{th} from the 0 hour and percentage of activity was also calculated. The changes in paw edema (ml) values were expressed as mean±S.E.M. The treatment values were compared with the control values for a particular hour using One-Way Analysis of Variance followed by Dunnett's multiple comparison test. Values of p<0.05 were considered as statistically significant. Nimesulide at 1, 3 and 10 mg/kg, p.o. showed dose dependant reduction in paw volume from 2 to 5 hour (table-1 and FIG. 1). However this was not observed at 30 mg/kg, wherein the degree of anti-inflammatory activity was around 30% from 2-5 hour (table-2 and FIG. 2). Nimesulide at 3 and 10 mg/kg at 3 hours and Nimesulide at 10 mg/kg at 5 hours showed statistically significant reduction in paw volume when compared to control. It was concluded that nimesulide at a dose of 10 mg/kg showed almost consistent anti-inflammatory activity at all time intervals.

TABLE 1
Effect of nimesulide in change in paw edema in carrageenan paw edema
model in rat.

| Treatment | Dose | Change in volume (ml) at hour (h) | | | | |
|---|---|---|---|---|---|---|
| (n = 6) | (mg/kg; p.o.) | 0.5 h | 1 h | 2 h | 3 h | 5 h |
| Control | — | 0.176 ± 0.028 | 0.173 ± 0.016 | 0.292 ± 0.036 | 0.494 ± 0.055 | 0.635 ± 0.047 |
| Nimesulide | 1 | 0.148 ± 0.033 | 0.186 ± 0.042 | 0.312 ± 0.081 | 0.398 ± 0.092 | 0.549 ± 0.114 |
| | 3 | 0.137 ± 0.038 | 0.126 ± 0.028 | 0.196 ± 0.056 | 0.168 ± 0.11* | 0.354 ± 0.104* |
| | 10 | 0.118 ± 0.027 | 0.143 ± 0.016 | 0.110 ± 0.018 | 0.203 ± 0.032* | 0.264 ± 0.035** |
| | 30 | 0.126 ± 0.027 | 0.193 ± 0.024 | 0.214 ± 0.028 | 0.307 ± 0.020 | 0.443 ± 0.035 |

Values are mean ± S.E.M.;
n = number of rats per group;
*p < 0.05;
**p < 0.001 vs. control;
One-Way ANOVA followed by Dunnett's multiple comparison test.

TABLE 2
Percentage of anti-inflammatory activity of nimesulide in
carrageenan paw edema model in rat.

| | | Percentage of anti-inflammatory | | | | |
|---|---|---|---|---|---|---|
| Treatment | Dose | activity at hour (h) | | | | |
| (n = 6) | (mg/kg; p.o.) | 0.5 h | 1 h | 2 h | 3 h | 5 h |
| Nimesulide | 1 | 16 | −8 | −7 | 20 | 14 |
| | 3 | 22 | 27 | 33 | 66* | 44 |
| | 10 | 33 | 17 | 62 | 59* | 58** |
| | 30 | 28 | −12 | 27 | 38 | 30 |

For reference, the p values are mentioned from Table 1 data.
*p < 0.05;
**p < 0.001 vs. control;
One-Way ANOVA followed by Dunnett's multiple comparison test.

Another pharmacological study has been carried out to study the analgesic activity of nimesulide on two mice models. Male Swiss mice (18-22 g) were selected for the study comprising 6 animals in each group. The dose volume was 0.1 ml/10 g body weight of mice and the route of administration was per oral (p.o.). Two tests were performed for the purpose. In Acetic acid induced writhing test, the 2 hour fasted mice were administered with various doses of nimesulide (0.03, 0.1, 0.3, 1 mg/kg). One hour later, a 1% acetic acid solution (10 ml/kg, i.p.) was used to produce writhing in mice. The numbers of writhes {constriction of abdomen, turning of trunk (twist), and extension of hind limbs} due to acetic acid were expressed as painful response. The numbers of writhes per animal were counted during a 20-minute session, beginning 3 minutes after the acetic acid injection. In second test i.e. Tail flick test, the mouse tail was placed on the radiant heat produced by the tail flick apparatus and the individual animal tail flick response was noted (a trail of three was carried out). Animals having a latency to tail withdrawal from the radiant heat source (3-5 s) were selected for the study. The selected mice were grouped and nimesulide (0.1, 0.3, 1, 3 mg/kg) was administered to the 2 hour fasted animals. 1, 2, 4, 5 hours later, the tail flick latency were noted and the change in latency and % maximum protective effect (% MPE) was calculated. A cut off time of 10 seconds was used to prevent any injury to the tail. The changes in tail flick latency and the number of writhes are expressed as mean±S.E.M. The treatment values were compared with the control values for a particular hour using One-Way Analysis of Variance followed by Dunnett's or Bonferroni's multiple comparison test. Values of p<0.05 were considered as statistically significant. Nimesulide at the doses 0.03, 0.1, 0.3 and 1 ml/kg showed dose dependant reduction in number of writhes induced by 1% acetic acid in mice (table-3 and FIG. 3). The doses 0.1, 0.3 and 1 mg/kg showed statistically significant reduction in number of writhing and the % of protection was 39, 52 and 75 respectively. In tail flick assay, the different doses (0.1, 0.3, 1 and 3 mg/kg) showed non dose dependant reduction in tail flick latency to radiant heat at all the time intervals (1, 2, 3 and 5 hours) tested (table-4 and FIG. 4). The percentage of maximum protection was also statistically significant at all time intervals and at all dose levels (table-5). The results indicate that nimesulide has analgesic activity even at low dose (0.3 mg/kg dose showed about 50% protection in acetic acid writhing test). The results were more consistent in Acetic acid writhing model than in Tail flick assays reflecting the dominant peripheral analgesic activity rather than analgesic activity mediated through central mechanism.

TABLE 3
Effect of nimesulide on 1% acetic acid induced writhing

| | Dose | | | |
|---|---|---|---|---|
| Treatment | (mg/kg; p.o) | N | No. of writhes | % protection |
| Control | — | 9 | 32.00 ± 1.39 | — |
| Nimesulide | 0.03 | 6 | 23.17 ± 3.38 | 28 |
| | 0.1 | 6 | 19.67 ± 2.73** | 39 |
| | 0.3 | 6 | 15.50 ± 1.88*** | 52 |
| | 1.0 | 6 | 8.00 ± 1.15*** | 75 |

Values are mean ± S.E.M;
N = number of rats per group;
**p < 0.01;
***p < 0.001 vs. control;
One-Way ANOVA followed by Bonferroni's multiple comparison test.

TABLE 4
Effect of nimesulide in tail flick assay

| Treatment | Dose | Tail flick latency(s) at hour (h) | | | |
|---|---|---|---|---|---|
| (N = 6) | (mg/kg; p.o) | 1 h | 2 h | 3 h | 5 h |
| Control | — | 0.00 ± 0.18 | −0.08 ± 0.24 | 0.25 ± 0.21 | −0.33 ± 0.11 |
| Nimesulide | 0.1 | 3.00 ± 0.58** | 5.50 ± 0.18** | 6.17 ± 0.33** | 1.42 ± 0.35* |
| | 0.3 | 3.17 ± 0.51** | 5.33 ± 0.38** | 5.75 ± 0.21** | 3.08 ± 0.27** |
| | 1.0 | 3.17 ± 0.67** | 5.25 ± 0.25** | 5.25 ± 0.28** | 2.17 ± 0.28** |
| | 3.0 | 5.17 ± 0.73** | 6.33 ± 0.33** | 6.25 ± 0.21** | 5.33 ± 0.85** |

Values are mean ± S.E.M; N = number of rats per group;
*p < 0.05,
**p < 0.01 vs. control;
One-Way ANOVA followed by Dunnett's multiple comparison test.

TABLE 5
Maximum percentage effect (analgesic) of nimesulide in tail flick assay

| Treatment | Dose | % MPE at hour (h) | | | |
|---|---|---|---|---|---|
| (N = 6) | (mg/kg; p.o) | 1 h | 2 h | 3 h | 5 h |
| Control | — | 1.0 ± 6.4 | −3.0 ± 8.2 | 8.0 ± 7.5 | −11.0 ± 3.6 |
| Nimesulide | 0.1 | 137.0 ± 37.1** | 251.0 ± 59.9** | 279.0 ± 49.4** | 28.0 ± 6.1** |
| | 0.3 | 114.0 ± 16.8* | 193.0 ± 9.5** | 209.0 ± 4.3** | 54.0 ± 6.6** |
| | 1.0 | 112.0 ± 22.5* | 187.0 ± 11.9** | 187.0 ± 12.0** | 38.0 ± 6.1** |
| | 3.0 | 205.0 ± 32.9** | 250.0 ± 21.9** | 248.0 ± 20.4** | 70.0 ± 9.5** |

Values are mean ± S.E.M;
N = number of rats per group;
*p < 0.05,
**p < 0.01 vs. control;
One-Way ANOVA followed by Dunnett's multiple comparison test.

### DESCRIPTION OF FIGURES

FIG. 1: The said figure shows the effect of nimesulide on carrageenan induced paw edema volume in right hind paw of the rats. Each bar represents mean±S.E.M. of 6 rats. *p<0.05; **p<0.001 vs. control; One-Way ANOVA followed by Dunnett's multiple comparison test.

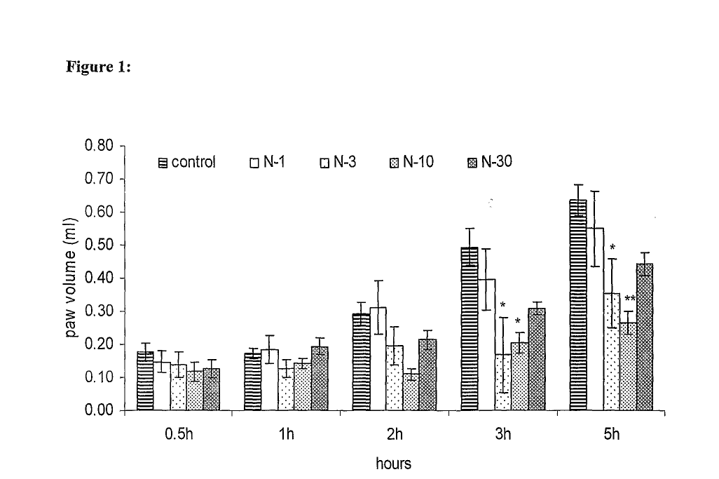

FIG. 2: The said figure shows the percentage of anti-inflammatory activity of nimesulide in carrageenan induced paw edema in rats.

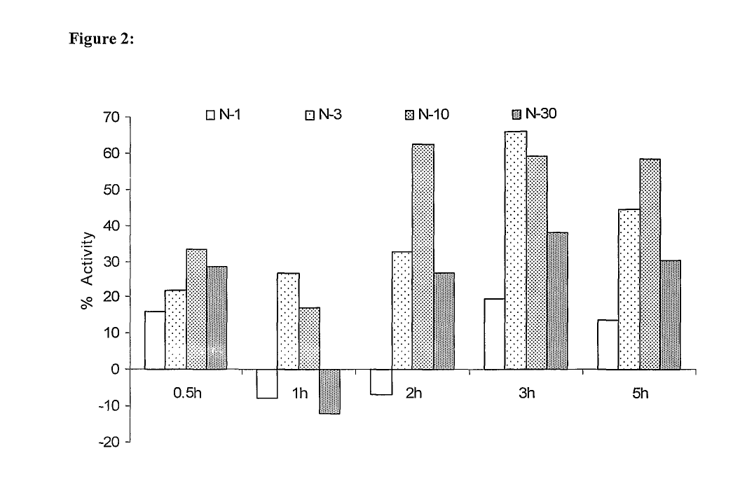

FIG. 3: The said figure shows the effect of nimesulide on 1% acetic acid induced writhing in mice. Each bar represents mean±S.E.M. of 6-9 mice. **p<0.01; ***p<0.001 vs. control; One-Way ANOVA followed by Bonferroni's multiple comparison test.

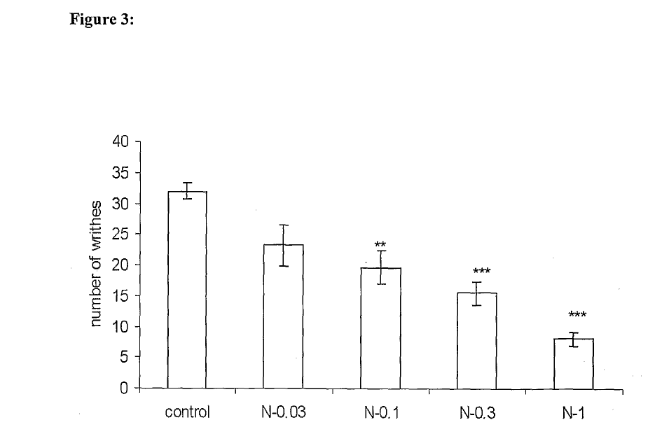

FIG. 4: The said figure shows the effect of nimesulide on tail flick latency induced by radiant heat. Each bar represents mean±S.E.M. of 6 mice. *p<0.05; **p<0.01 vs. control; One-Way ANOVA followed by Dunnett's multiple comparison test.

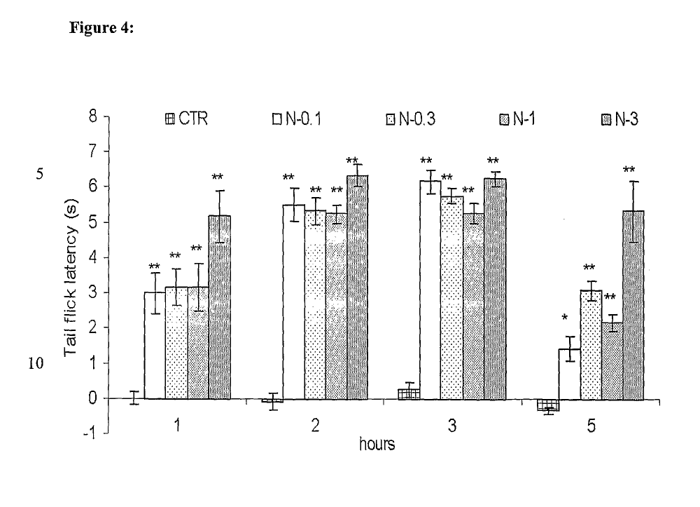

The following examples are only intended to further illustrate different embodiments of the invention, and are therefore not deemed to restrict the scope of the invention in any way.

### EXAMPLES

### Example-1

### Tablet

| S. No. | Ingredient | Quantity/tablet (mg) |
|---|---|---|
| 1. | Nimesulide | 75.0 |
| 2. | Microcrystalline cellulose | 285.0 |
| 3. | Lactose | 100.0 |
| 4. | Croscarmellose sodium | 20.0 |
| 5. | Isopropyl alcohol | q.s. (lost in |
| | | processing) |
| 6. | Hydrogenated castor oil | 7.5 |
| 7. | Purified talc | 7.5 |
| 8. | Colloidal silicon dioxide | 7.5 |

### Procedure:

i) Nimesulide, Lactose, Microcrystalline cellulose and Croscarmellose sodium were sifted through #40 sieve and were mixed together.
ii) The blend of step (i) was granulated using Isopropyl alcohol.
iii) The wet mass of step (ii) was sifted through #24 sieve and granules obtained were dried.
iv) Hydrogenated castor oil, Purified talc and Colloidal silicon dioxide were sifted through #40 sieve and were mixed together.
v) Granules of step (iii) were mixed with the mixture of step (iv).
vi) The material of step (v) was compressed into tablets by using a tablet compression machine.

### Example-2

### Tablet

| S. No. | Ingredient | Quantity/tablet (mg) |
|---|---|---|
| 1. | Nimesulide | 50.0 |
| 2. | Mannitol | 80.0 |
| 3. | Sodium starch glycollate | 5.0 |
| 4. | Colloidal silicon dioxide | 3.0 |
| 5. | Corn starch | 10.0 |
| 6. | Povidone (K-30) | 3.0 |
| 7. | Sodium lauryl sulphate | 1.0 |
| 8. | Purified water | q.s. (lost in |
| | | processing) |
| 9. | Magnesium stearate | 1.0 |
| 10. | Croscarmellose sodium | 8.0 |

### Procedure

i) Nimesulide, Mannitol, Sodium starch glycollate, Colloidal silicon dioxide and Corn starch were mixed together and sifted through mesh #30 sieve.
ii) Povidone (K-30) and Sodium lauryl sulphate were dissolved in Purified water to obtain a homogeneous solution.
iii) The material of step (i) was granulated with the material of step (ii) followed by drying and sifting through mesh #16 sieve.
iv) Magnesium stearate and Croscarmellose sodium were sifted through mesh #40 sieve.
v) The material of step (iv) was mixed with the material of step (iii).

### Example-3

### Capsule (Hard Gelatin)

| S. No. | Ingredient | Quantity/capsule (mg) |
|---|---|---|
| 1. | Nimesulide | 25.00 |
| 2. | Magnesium carbonate | 150.00 |
| 3. | Dicalcium phosphate | 131.25 |
| 4. | Crospovidone | 30.00 |
| 5. | Magnesium stearate | 10.00 |

### Procedure:

i) Nimesulide, Magnesium carbonate, Dicalcium phosphate, Crospovidone, and Magnesium stearate were sifted through #40 sieve and were mixed together.
ii) The blend of step (i) was compacted and the compacts were passed through #30 sieve.
iii) The granules of step (ii) were lubricated with #60 sieve passed Magnesium stearate.
iv) The material of step (iii) was filled into hard gelatin capsule.

### Example-4

### Modified Release Tablet

| S. No. | Ingredient | Quantity/tablet (mg) |
|---|---|---|
| 1. | Nimesulide | 75.0 |
| 2. | Cetirizine | 2.0 |
| 3. | Mannitol | 49.0 |
| 4. | Croscarmellose sodium | 10.0 |
| 5. | Hydroxypropyl methylcellulose | 20.0 |
| 6. | Isopropyl alcohol | q.s. (lost in |
| | | processing) |
| 7. | Colloidal silicon dioxide | 2.0 |
| 8. | Hydrogenated vegetable oil | 2.0 |

### Procedure:

i) Nimesulide, Cetirizine, Mannitol and Croscarmellose sodium were sifted through #30 sieve and were mixed together.
ii) Hydroxypropyl methylcellulose was dissolved in Isopropyl alcohol to obtain a homogeneous dispersion.
iii) The blend of step (i) was granulated with the dispersion of step (ii).
iv) The granules of step (iii) were dried and were sifted through #24 sieve.
v) Colloidal silicon dioxide and Hydrogenated vegetable oil were sifted through #40 sieve.
vi) The material of step (v) was mixed with the material of step (iv) and compressed into tablets.

### Example-5

### Capsule (Hard Gelatin)

| | | Quantity/ |
|---|---|---|
| S. No. | Ingredient | tablet (mg) |
| 1. | Nimesulide | 25.0 |
| 2. | Propylene glycol | 108.0 |
| 3. | Poly Oxyl 40 Hydrogenated Castor Oil | 10.0 |
| | (Cremophor ® RH 40) | |
| 4. | Propylene glycol laurate | 130.0 |

### Procedure:

i) Propylene glycol was mixed with Cremophor® RH 40 and heated up to 55 to 60° C. and Nimesulide was dissolved in the resultant mixture.
ii) Propylene glycol laurate was then added to the bulk mixture of step (i) and mixed. The resultant mixture was then filtered.
iii) The mixture of step (ii) was filled into hard gelatin capsules and sealed.

### Example-6

### Capsule (Soft Gelatin)

| | | Quantity/ |
|---|---|---|
| S. No. | Ingredient | capsule (mg) |
| 1. | Nimesulide | 20.0 |
| 2. | Propylene Glycol | 85.0 |
| 3. | Cremophor ® RH 40 | 5.0 |
| 4. | Propylene glycol laurate | 107.0 |
| 5. | Propylene glycol dicaprylate/dicaprate | 5.0 |
| 6. | Triacetin | 1.5 |

### Procedure:

i) Propylene Glycol was mixed with Cremophor® RH 40 and heated up to 55 to 60° C. and Nimesulide was dissolved in the resultant mixture.
ii) Propylene glycol laurate was then added to the bulk mixture of step (i) and mixed.
iii) Propylene glycol dicaprylate/dicaprate followed by Triacetin was added to the mixture of step (ii). The resultant mixture was then filtered.
iv) The mixture of step (iii) was filled into soft gelatin capsules.

### Example-7

### Injection

| | | Quantity/ | |
|---|---|---|---|
| S. No. | Ingredient | 100 ml | |
| 1. | Polyethylene glycol (PEG-400) | 30.0 | ml |
| 2. | Propylene glycol | 20.0 | ml |
| 3. | Glycine buffer pH 11.3 | 35.0 | ml |
| 4. | Nimesulide | 1.0 | g |
| 5. | Sodium hydroxide (NaOH) | 10.0 | ml |
| | solution 4.0% w/v | | |

### Procedure:

i) Specified quantity (30.0 ml) of PEG-400 was taken into a vessel.
ii) Propylene glycol (20.0 ml) was added to step (i) with continuous stirring using mechanical stirrer.
iii) About 30.0 ml of the Glycine buffer pH 11.3 was added to the step (ii) with continuous stirring to form a homogeneous mixture.
iv) Weighed amount of Nimesulide (1.0 g) was passed through #60 sieve and was added to the step (iii) with continuous stirring.
v) Specified quantity (10.0 ml) of Sodium hydroxide (NaOH) 4.0% w/v solution was added to the step (iv) with continuous stirring to form a homogeneous solution.
vi) The solution of step (v) was mixed for about 30 minutes by continuous stirring.
vii) Remaining quantity of Glycine Buffer pH 11.3 was added to make up volume to 100 ml.
viii) The solution of step (vii) was mixed for about 10 minutes by continuous stirring.
ix) Final pH was adjusted to 10.0 by adding Sodium hydroxide (NaOH) 4.0% w/v solution.
x) The solution of step (ix) was mixed for about 10 minutes by continuous stirring.

### Example-8

### Oral Suspension

| | | Quantity |
|---|---|---|
| S. No. | Ingredient | (mg/5 ml) |
| 1. | Nimesulide | 40.0 |
| 2. | Citric acid monohydrate | 1.5 |
| 3. | Hydroxyethyl cellulose | 20.0 |
| 4. | Sorbitol solution (70% w/v) | 50.0 |
| 5. | Saccharin sodium | 0.5 |
| 6. | Sodium benzoate | 1.0 |
| 7. | Raspberry flavor | q.s. |
| 8. | Purified water | q.s. to 5 ml |

### Procedure:

i) Nimesulide and Hydroxyethyl cellulose were sifted through #40 sieve and were blended together.
ii) Citric acid monohydrate, Saccharin sodium, Sodium benzoate, Raspberry flavor and Sorbitol solution were dispersed together in Purified water.
iii) The material of step (i) was added with continuous stirring to the material of step (ii) and a homogeneous suspension was obtained.

### Example-9

### Nimesulide Modified Release Minitablets Filled in Capsule

### A) Immediate Release Fraction

| S. No. | Ingredient | Quantity (mg) |
|---|---|---|
| 1. | Nimesulide | 25.0 |
| 2. | Mannitol | 10.0 |
| 3. | Sodium starch glycollate | 8.0 |
| 4. | Corn starch | 5.0 |
| 5. | Polysorbate 80 | 1.0 |
| 6. | Magnesium stearate | 1.0 |

### Procedure:

i) Nimesulide, Mannitol, Sodium starch glycollate, Corn starch and Polysorbate 80 were mixed together and sifted through mesh #30 sieve.
ii) Magnesium stearate was sifted through mesh #40 sieve.
iii) Material of step (i) was mixed with material of step (ii) and compressed into mini-tablet.

### B) Delayed Release Fraction:

| S. No. | Ingredient | Quantity (mg) |
|---|---|---|
| 1. | Nimesulide | 25.0 |
| 2. | Lactose monohydrate | 6.5 |
| 3. | Docusate sodium | 2.0 |
| 4. | Povidone (K-30) | 3.0 |
| 5. | Colloidal silicon dioxide | 3.0 |
| 6. | Magnesium stearate | 3.0 |
| 7. | Methacrylate polymer | 5.5 |
| 8. | Triethyl citrate | 1.5 |
| 9. | Isopropyl alcohol | q.s. (lost in processing) |
| 10. | Methylene chloride | q.s. (lost in processing) |

### Procedure:

i) Nimesulide, Lactose monohydrate, Docusate sodium, Povidone (K-30) and Colloidal silicon dioxide were mixed together and sifted through mesh #30 sieve.
ii) Magnesium stearate was sifted through mesh #40 sieve.
iii) The material of step (i) was mixed with material of step (ii) and compressed into minitablet.
iv) Methacrylate polymer and Triethyl citrate were dispersed in a mixture of Isopropyl alcohol and Methylene chloride and mixed.
v) The minitablets of step (iii) were coated with material of step (iv).

### C) Sustained Release Fraction:

| S. No. | Ingredient | Quantity (mg) |
|---|---|---|
| 1. | Nimesulide | 50.0 |
| 2. | Lactose monohydrate | 8.0 |
| 3. | Sodium carboxymethylcellulose | 6.0 |
| 4. | Docusate sodium | 2.0 |
| 5. | Povidone (K-30) | 2.0 |
| 6. | Purified water | q.s. (lost in |
| | | processing) |
| 7. | Colloidal silicon dioxide | 3.0 |
| 8. | Magnesium Stearate | 3.0 |

### Procedure:

i) Nimesulide, Lactose monohydrate, Sodium carboxymethylcellulose were mixed together and sifted through mesh #30 sieve.
ii) Docusate sodium and Povidone (K-30) were dissolved in water to form a homogeneous dispersion.
iii) The material of step (i) was granulated with material of step (ii) followed by drying and sifting through mesh #18 sieve.
iv) Colloidal silicon dioxide and Magnesium Stearate were sifted through mesh#40 sieve.
v) The material of step (iii) was mixed with material of step (iv) and compressed into minitablets.

The minitablets obtained in step (iii) of (A), step (v) of (B) & (C) were filled into hard gelatin capsule.

### Example-10

### Nimesulide Gel

| S. No. | Ingredient | Quantity (g/100 gm) |
|---|---|---|
| 1. | Nimesulide | 0.5 |
| 2. | Dimethylacetamide | 10.0 |
| 3. | Ethyl alcohol | 20.0 |
| 4. | Acetone | 5.0 |
| 5. | Cremophor ® RH40 | 1.0 |
| 6. | Propylene glycol | 20.0 |
| 7. | Carbopol 934 | 1.2 |
| 8. | Purified water | 20 |
| 9. | Diethylamine | 0.6 |

### Procedure:

i) Dimethylacetamide was mixed with Ethyl alcohol and acetone in a container with stirring.
ii) To the mixture obtained, nimesulide was added and stirred till completely dissolved.
iii) Propylene glycol and Cremophor® RH40 were added to Purified water and were mixed in homogenizer. To the homogenised mixture obtained, Carbopol 934 was added and further homogenized.
iv) The mixture obtained in step (ii) was added to the mixture obtained in step (iii) under stirring.
v) The mixture obtained was neutralized by slow addition of Diethylamine with slow stirring to produce the desired gel.

### Example-11

### Controlled Release Matrix Tablet Type

| S. No. | Ingredient | Quantity/tablet (mg) |
|---|---|---|
| 1. | Nimesulide | 180 |
| 2. | Lactose | 80 |
| 3. | Hydroxypropylmethyl Cellulose | 80 |
| 4. | Magnesium Stearate | 5 |
| 5. | Purified Talc | 5 |

### Procedure:

i) Nimesulide Lactose, Hydroxypropylmethyl Cellulose, Magnesium Stearate, and Purified Talc were sifted through mesh # 30 (BSS) sieve;
ii) The material of step (i) was blended together; and
iii) The mixture obtained from step (ii) is compressed into tablets.

### Example-12

### Extended Release Membrane Diffusion Controlled Tablet Type

| S. No. | Ingredient | Quantity/tablet (mg) |
|---|---|---|
| 1. | Nimesulide | 125 |
| 2. | Mycrocrystalline Cellulose | 80 |
| 3. | Lactose | 80 |
| 4. | Maize Starch | 10 |
| 5. | Purified Talc | 3.5 |
| 6. | Ethyl cellulose | 10 |
| | (As aqueous Dispersion) | |
| 7. | Polyethylene glycol | 3.5 |

### Procedure:

i) Nimesulide, Microcrystalline Cellulose and Lactose were granulated with starch paste.
ii) The granules of step (i) were sifting through mesh # 22 (BSS).
iii) The sifted granules were dried and lubricated with purified talc.
iv) Compress the dried granules into tablets.
v) Ethylcellulose and polyethylene glycol dispersion was prepared and the tablet of step (iv) were coated.

1. A novel low dose pharmaceutical dosage form comprising nimesulide or its pharmaceutically acceptable salts, esters, prodrugs, solvates, hydrates, or derivatives thereof, with one or more pharmaceutically acceptable excipient(s).

2. The novel low dose pharmaceutical dosage form according to claim 1, wherein the total daily dose of nimesulide is less than the conventionally administered daily dose of at least about 200 mg of nimesulide.

3. The novel low dose pharmaceutical dosage form according to claim 2, wherein the dose of the nimesulide is in an amount below 200 mg, intended for once-a-day administration.

4. The novel low dose pharmaceutical dosage form according to claim 3, wherein the dose of the nimesulide is in an amount ranges form about 125 mg to about 180 mg, intended for once-a-day administration.

5. The novel low dose pharmaceutical dosage form according to claim 3, wherein the low dose of the nimesulide intended for once-a-day administration is administered either in single unit or in a multiple unit.

6. The novel low dose pharmaceutical dosage form according to claim 5, wherein the low dose of the nimesulide intended for once-a-day administration is administered in a single unit, preferably in the form of tablet.

7. The novel low dose pharmaceutical dosage form according to claim 2, wherein the individual dose of the nimesulide is in an amount below 100 mg, for a single administration intended for twice-a-day administration.

8. The novel low dose pharmaceutical dosage form according to claim 7, wherein the individual dose of the nimesulide is in an amount ranges from about 25 mg to about 85 mg, for a single administration intended for twice-a-day administration.

9. The novel low dose pharmaceutical dosage form according to claim 1, wherein the pharmaceutically acceptable excipient(s) is selected amongst polymeric material, gum, surfactant, complexing agent, diluents, disintegrants, binders, mucoadhesive agents, fillers, bulking agents, anti-adherants, anti-oxidants, buffering agents, colorants, flavoring agents, coating agents, plasticizers, stabilizers, preservatives, lubricants, glidants, chelating agents, used either alone or in combination.

10. The novel low dose pharmaceutical dosage form according to claim 1, wherein the dosage form additionally comprises one or more other active agent(s) selected amongst antipyretics, antiallergics, aldosterone receptor antagonists, antibiotics, various enzymes, antimuscarinic agents, anti-viral agents, protein kinase inhibitors, β2-adrenergic agonist, ACE inhibitors, opoid analgesics, steroids, leukotriene B4(LTB4) receptor antagonists, leukotriene A4 (LTA4) hydrolase inhibitors, 5-HT agonists, HMG CoA inhibitors, H2 antagonists, antineoplastic agents, antiplatelet agents, thrombin inhibitors, decongestants, diuretics, sedating or non-sedating anti-histamines, inducible nitric oxide synthase inhibitors, opioids, analgesics, Helicobacter pylori inhibitors, bronchodilators, spasmolytics, muscle relaxants, proton pump inhibitors, isoprostane inhibitors, PDE4-inhibitors, other NSAIDs, selective or preferential COX-2 inhibitors, COX-1 inhibitors, expectorants, analgesics, antiemetics, urinary acidifiers, antidepressants, antipsychotics, antimigraine agents, and the like or mixtures.

11. The novel low dose pharmaceutical dosage form according to claim 1, wherein the dosage form is selected amongst tablets, capsules, liquid dispersions, oral suspensions, gels, aerosols, ointments, creams, fast melt formulations, lyophilized formulations, injectables, controlled release formulations, delayed release formulations, extended release formulations, pulsatile release formulations, and a combination of immediate release and controlled release formulations.

12. The novel low dose pharmaceutical dosage form according to claim 11, wherein the dosage form is in the form of tablet, which may be coated with one or more functional or non-functional coating layers.

13. The novel low dose pharmaceutical dosage form according to claim 1, wherein the dosage form is prepared by a process comprises of the following steps:
i) treating nimesulide with one or more pharmaceutically acceptable excipient(s),
ii) optionally adding one or more other active agent(s), and
iii) formulating into a suitable dosage form.

14. The novel low dose pharmaceutical dosage form according to claim 1, wherein the dosage form is capable of providing therapeutically effective bioavailability of nimesulide with reduced side effects, after dosing in a human subject.

15. The novel low dose pharmaceutical dosage form according to claim 1, wherein the dosage form is useful in the management of cyclooxygenase enzyme mediated disorders and/or cyclooxygenase inhibitor indicated disorders.

16. The method of using a novel low dose pharmaceutical dosage form according to claim 1, for the treatment of NSAID indicated disorders, early morning pathologies, post-operative trauma, pain associated with cancer, postoperative pain, sports injuries, dysmenorrhoea, migraine headache, neurological pain and pain associated with sciatica and spondylitis, arthritis, idiopathic pain, myofascial pain, osteoarthitis, neuropathic pain, fibromyalgia and inflammatory pain states; cancer pain; chronic pain; sympathetically mediated pain, Raynaud's disease, CPS (Chronic Pain Syndrome); tension and migraine headache, stump pain, polyarteritis nodosa, osteomyelitis, burns involving nerve damage, AIDS related pain syndromes, and connective tissue disorders, other degenerative joint disorders, or any other disorders mediated by particularly the cyclooxygenase enzyme, pain and/or inflammation associated with osteoarthritis, ligamentous pain, bursitis, tendinitis, low back pain, postoperative pain, dental extraction or surgery; saphenectomy or inguinal hemioplasty; haemorrhoidectomy; acute musculoskeletal injury; ear, nose or throat disorders; gynaecological disorders; cancer pain; alzheimer's disease; thrombophlebitis; urogenital disorders; bursitis or tendonitis; morning stiffness associated with rheumatoid arthritis, shingles, trigeminal neuralgia, temporomandibular joint disorder; cancer pain or a combination of several disorders or any other associated disorder.

17. (canceled)
